(19) United States
(12) Patent Application Publication
(10) Pub. No.: US 2018/0044375 A1
(43) Pub. Date: Feb. 15, 2018

(54) DEUTERATED EMRICASAN

(71) Applicant: Concert Pharmaceuticals, Inc., Lexington, MA (US)

(72) Inventor: Scott L. Harbeson, Cambridge, MA (US)

(21) Appl. No.: 15/555,471

(22) PCT Filed: Mar. 4, 2016
(86) PCT No.: PCT/US2016/021071

§ 371 Date: Sep. 1, 2017

Related U.S. Application Data
(60) Provisional application No. 62/129,332, filed on Mar. 6, 2015.

(51) Int. Cl. C07K 5/062

(52) U.S. Cl. CPC C07K 5/06026; C07B 2200/05; A61K 38/00

(57) ABSTRACT

This invention relates to novel 3-amido-4-oxo-5-phenoxypentanoic acids, and pharmaceutically acceptable salts thereof. This invention also provides compositions comprising a compound of this invention and the use of such compositions in methods of treating diseases and conditions that are beneficially treated by administering a caspase protease inhibitor.

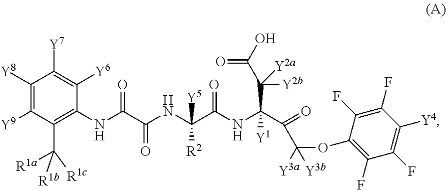

### BACKGROUND OF THE INVENTION

Many current medicines suffer from poor absorption, distribution, metabolism and/or excretion (ADME) properties that prevent their wider use or limit their use in certain indications. Poor ADME properties are also a major reason for the failure of drug candidates in clinical trials. While formulation technologies and prodrug strategies can be employed in some cases to improve certain ADME properties, these approaches often fail to address the underlying ADME problems that exist for many drugs and drug candidates. One such problem is rapid metabolism that causes a number of drugs, which otherwise would be highly effective in treating a disease, to be cleared too rapidly from the body. A possible solution to rapid drug clearance is frequent or high dosing to attain a sufficiently high plasma level of drug. This, however, introduces a number of potential treatment problems such as poor patient compliance with the dosing regimen, side effects that become more acute with higher doses, and increased cost of treatment. A rapidly metabolized drug may also expose patients to undesirable toxic or reactive metabolites.

Another ADME limitation that affects many medicines is the formation of toxic or biologically reactive metabolites. As a result, some patients receiving the drug may experience toxicities, or the safe dosing of such drugs may be limited such that patients receive a suboptimal amount of the active agent. In certain cases, modifying dosing intervals or formulation approaches can help to reduce clinical adverse effects, but often the formation of such undesirable metabolites is intrinsic to the metabolism of the compound.

In some select cases, a metabolic inhibitor will be co-administered with a drug that is cleared too rapidly. Such is the case with the protease inhibitor class of drugs that are used to treat HIV infection. The FDA recommends that these drugs be co-dosed with ritonavir, an inhibitor of cytochrome P450 enzyme 3A4 (CYP3A4), the enzyme typically responsible for their metabolism (see Kempf, D. J. et al., Antimicrobial agents and chemotherapy, 1997, 41(3): 654-60). Ritonavir, however, causes adverse effects and adds to the pill burden for HIV patients who must already take a combination of different drugs. Similarly, the CYP2D6 inhibitor quinidine has been added to dextromethorphan for the purpose of reducing rapid CYP2D6 metabolism of dextromethorphan in a treatment of pseudobulbar affect. Quinidine, however, has unwanted side effects that greatly limit its use in potential combination therapy (see Wang, L et al., Clinical Pharmacology and Therapeutics, 1994, 56(6 Pt 1): 659-67; and FDA label for quinidine at www.accessdata.fda.gov).

In general, combining drugs with cytochrome P450 inhibitors is not a satisfactory strategy for decreasing drug clearance. The inhibition of a CYP enzyme's activity can affect the metabolism and clearance of other drugs metabolized by that same enzyme. CYP inhibition can cause other drugs to accumulate in the body to toxic levels.

A potentially attractive strategy for improving a drug's metabolic properties is deuterium modification. In this approach, one attempts to slow the CYP-mediated metabolism of a drug or to reduce the formation of undesirable metabolites by replacing one or more hydrogen atoms with deuterium atoms. Deuterium is a safe, stable, non-radioactive isotope of hydrogen. Compared to hydrogen, deuterium forms stronger bonds with carbon. In select cases, the increased bond strength imparted by deuterium can positively impact the ADME properties of a drug, creating the potential for improved drug efficacy, safety, and/or tolerability. At the same time, because the size and shape of deuterium are essentially identical to those of hydrogen, replacement of hydrogen by deuterium would not be expected to affect the biochemical potency and selectivity of the drug as compared to the original chemical entity that contains only hydrogen.

Over the past 35 years, the effects of deuterium substitution on the rate of metabolism have been reported for a very small percentage of approved drugs (see, e.g., Blake, M I et al, J Pharm Sci, 1975, 64:367-91; Foster, A B, Adv Drug Res 1985, 14:1-40 (“Foster”); Kushner, D J et al, Can J Physiol Pharmacol 1999, 79-88; Fisher, M B et al, Curr Opin Drug Discov Devel, 2006, 9:101-09 (“Fisher”)). The results have been variable and unpredictable. For some compounds deuteration caused decreased metabolic clearance in vivo. For others, there was no change in metabolism. Still others demonstrated increased metabolic clearance. The variability in deuterium effects has also led experts to question or dismiss deuterium modification as a viable drug design strategy for inhibiting adverse metabolism (see Foster at p. 35 and Fisher at p. 101).

The effects of deuterium modification on a drug's metabolic properties are not predictable even when deuterium atoms are incorporated at known sites of metabolism. Only by actually preparing and testing a deuterated drug can one determine if and how the rate of metabolism will differ from that of its non-deuterated counterpart. See, for example, Fukuto et al. (J. Med. Chem. 1991, 34, 2871-76). Many drugs have multiple sites where metabolism is possible. The site(s) where deuterium substitution is required and the extent of deuteration necessary to see an effect on metabolism, if any, will be different for each drug.

### SUMMARY OF THE INVENTION

This invention relates to novel 3-amido-4-oxo-5-phenoxypentanoic acids, and pharmaceutically acceptable salts thereof. This invention also provides compositions comprising a compound of this invention and the use of such compositions in methods of treating diseases and conditions that are beneficially treated by administering a caspase protease inhibitor.

Emricasan, also known as IDN 6556, PF 03491390, and (3S)-3-[N-(N′-(2-tert-butylphenyl)oxamyl)alaninyl]amino-5-(2′,3′,5′,6′-tetrafluorophenoxy)-4-oxopentanoic acid, modulates interleukin-1β converting enzyme and related proteases (ICE/ced-3 family of cysteine proteases). In particular, emricasan modulates the activity of caspases.

Emricasan is in phase II clinical trials for the treatment of chronic Hepatitis C, hepatitis, liver transplantation, cholestatis, hepatocellular carcinoma, acute or chronic hepatic failure, acute liver failure, liver cirrhosis, acute alcoholic hepatitis, diabetes, liver fibrosis, hepatic fibrosis, non-alcoholic steatohepatitis, non-alcoholic fatty liver disease, alcoholic hepatitis, liver cirrhosis, hepatic cirrhosis and portal hypertension; phase I clinical trials for the treatment of hepatic impairment, liver diseases, gastrointestinal diseases, digestive system diseases, renal impairment, renal insufficiency, kidney diseases and urologic diseases; and is currently being tested for safety and efficacy in patients with acute exacerbation of the abovementioned diseases.

Despite the beneficial activities of emricasan, there is a continuing need for new compounds to treat the aforementioned diseases and conditions.

### DETAILED DESCRIPTION OF THE INVENTION

### Definitions

The term “treat” means decrease, suppress, attenuate, diminish, arrest, or stabilize the development or progression of a disease (e.g., a disease or disorder delineated herein), lessen the severity of the disease or improve the symptoms associated with the disease.

“Disease” means any condition or disorder that damages or interferes with the normal function of a cell, tissue, or organ.

It will be recognized that some variation of natural isotopic abundance occurs in a synthesized compound depending upon the origin of chemical materials used in the synthesis. Thus, a preparation of emricasan will inherently contain small amounts of deuterated isotopologues. The concentration of naturally abundant stable hydrogen and carbon isotopes, notwithstanding this variation, is small and immaterial as compared to the degree of stable isotopic substitution of compounds of this invention. See, for instance, Wada, E et al., Seikagaku, 1994, 66:15; Gannes, L Z et al., Comp Biochem Physiol Mol Integr Physiol, 1998, 119:725.

In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Also unless otherwise stated, when a position is designated specifically as “D” or “deuterium”, the position is understood to have deuterium at an abundance that is at least 3340 times greater than the natural abundance of deuterium, which is 0.015% (i.e., at least 50.1% incorporation of deuterium).

The term “isotopic enrichment factor” as used herein means the ratio between the isotopic abundance and the natural abundance of a specified isotope.

In other embodiments, a compound of this invention has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

The term “isotopologue” refers to a species in which the chemical structure differs from a specific compound of this invention only in the isotopic composition thereof.

The term “compound,” when referring to a compound of this invention, refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this invention will depend upon a number of factors including the isotopic purity of deuterated reagents used to make the compound and the efficiency of incorporation of deuterium in the various synthesis steps used to prepare the compound. However, as set forth above, the relative amount of such isotopologues in toto will be less than 49.9% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound.

The invention also provides salts of the compounds of the invention.

A salt of a compound of this invention is formed between an acid and a basic group of the compound, such as an amino functional group, or a base and an acidic group of the compound, such as a carboxyl functional group. According to another embodiment, the compound is a pharmaceutically acceptable acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfate, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

The pharmaceutically acceptable salt may also be a salt of a compound of the present invention having an acidic functional group, such as a carboxylic acid functional group, and a base. Exemplary bases include, but are not limited to, hydroxide of alkali metals including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

The compounds of the present invention (e.g., compounds of Formula A and/or Formula I), may contain an asymmetric carbon atom, for example, as the result of deuterium substitution or otherwise. As such, compounds of this invention can exist as either individual enantiomers, or mixtures of the two enantiomers. Accordingly, a compound of the present invention may exist as either a racemic mixture or a scalemic mixture, or as individual respective stereoisomers that are substantially free from another possible stereoisomer. The term “substantially free of other stereoisomers” as used herein means less than 25% of other stereoisomers, preferably less than 10% of other stereoisomers, more preferably less than 5% of other stereoisomers and most preferably less than 2% of other stereoisomers are present. Methods of obtaining or synthesizing an individual enantiomer for a given compound are known in the art and may be applied as practicable to final compounds or to starting material or intermediates.

Unless otherwise indicated, when a disclosed compound is named or depicted by a structure without specifying the stereochemistry and has one or more chiral centers, it is understood to represent all possible stereoisomers of the compound.

The term “stable compounds,” as used herein, refers to compounds which possess stability sufficient to allow for their manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to therapeutic agents).

“D” and “d” both refer to deuterium. “Stereoisomer” refers to both enantiomers and diastereomers. “Tert” and “t-” each refer to tertiary. “US” refers to the United States of America.

“Substituted with deuterium” refers to the replacement of one or more hydrogen atoms with a corresponding number of deuterium atoms.

Throughout this specification, a variable may be referred to generally (e.g.,“each R”) or may be referred to specifically (e.g., R¹, R², R³, etc.). Unless otherwise indicated, when a variable is referred to generally, it is meant to include all specific embodiments of that particular variable.

### Therapeutic Compounds

The present invention provides a compound of Formula A:

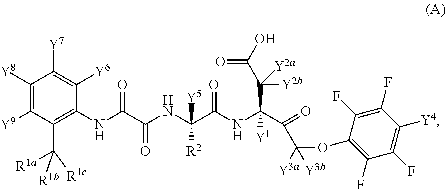

or a pharmaceutically acceptable salt thereof,
wherein each of R^{1a}, R^{1b}, R^{1c} and R² is independently selected from CH₃, CHD₂, CH₂D and CD₃;
and each of Y¹, Y^{2a}, Y^{2b}, Y^{3a}, Y^{3b}, Y⁴, Y⁵, Y⁶, Y⁷, Y⁸, and Y⁹ is independently hydrogen or deuterium;
provided that at least one of R^{1a}, R^{1b}, R^{1c}, R², Y¹, Y^{2a}, Y^{2b}, Y^{3a}, Y^{3b}, Y⁴, Y⁵, Y⁶, Y⁷, Y⁸ and Y⁹ comprises deuterium.

In some embodiments, R^{1a}, R^{1b} and R^{1c} are the same. In some aspects of these embodiments, R^{1a}, R^{1b} and R^{1c} are each CHD₂. In other aspects of these embodiments, R^{1a}, R^{1b} and R^{1c} and are each CH₂D. In yet other aspects of these embodiments, R^{1a}, R^{1b} and R^{1c} are each CD₃. In further aspects of these embodiments, R^{1a}, R^{1b} and R^{1c} are each CH₃.

In some embodiments, R^{1a} is CHD₂ and R^{1b}, R^{1c} are the same or different. In particular aspects of these embodiments, R^{1b} and R^{1c} comprise deuterium.

In some embodiments, R^{1b} is CHD₂ and R^{1a}, R^{1c} are the same or different. In particular aspects of these embodiments, R^{1a} and R^{1c} comprise deuterium.

In some embodiments, R^{1c} is CHD₂ and R^{1a}, R^{1bc} are same or different. In particular aspects of these embodiments, R^{1a} and R^{1c} comprise deuterium.

In some embodiments, R^{1a} is CH₂D and R^{1b}, R^{1c} are the same or different. In particular aspects of these embodiments R^{1b} and R^{1c} comprise deuterium.

In some embodiments, R^{1b} is CH₂D and R^{1a} R^{1c} are the same or different. In particular aspects of these embodiments R^{1b} and R^{1c} comprise deuterium.

In some embodiments, R^{1c} is CH₂D and R^{1b}, R^{1c} are the same or different. In particular aspects of these embodiments R^{1b} and R^{1c} comprise deuterium.

In some embodiments, R^{1a} is CD₃ and R^{1b}, R^{1c} are the same or different. In particular aspects of these embodiments R^{1b} and R^{1c} comprise deuterium.

In some embodiments, R² is CD₃.

In some embodiments, R² is CH₃.

In some embodiments, R² is CHD₂.

In some embodiments, R² is CH₂D.

In some embodiments, R^{1a}, R^{1b}, R^{1c} and R² are the same. In particular aspect of these embodiments, R^{1a}, R^{1b}, R^{1c} and R² are each CD₃.

In some embodiments, R^{1a}, R^{1b} and R^{1c} are all CH₃; and R² is CD₃.

In some embodiments, R^{1a}, R^{1b} and R^{1c} are all CH₃; and R² is CHD₂.

In some embodiments, R^{1a}, R^{1b} and R^{c} are all CH₃; and R² is CH₂D.

In some embodiments, R^{1a}, R^{1b} and R^{1c} are all CHD₂; and R² is CH₃.

In some embodiments, R^{1a}, R^{1b}, R^{1c} and R² are all CHD₂.

In some embodiments, R^{1a}, R^{1b} and R^{1c} are all CHD₂; and R² is CH₂D.

In some embodiments, R^{1a}, R^{1b} and R^{1c} are all CHD₂; and R² is CD₃.

In some embodiments, R^{1a}, R^{1b} and R^{1c} are all CH₂D; and R² is CH₃.

In some embodiments, R^{1a}, R^{1b} and R^{1c} are all CH₂D; and R² is CHD₂.

In some embodiments, R^{1a}, R^{1b}, R^{1c} and R² are all CH₂D.

In some embodiments, R^{1a}, R^{1b} and R^{1c} are all CH₂D; and R² is CD₃.

In some embodiments, R^{1a}, R^{1b} and R^{1c} are all CD₃; and R² is CH₃.

In some embodiments, R^{1a}, R^{1b} and R^{1c} are all CD₃; and R² is CHD₂.

In some embodiments, R^{1a}, R^{1b} and R^{1c} are all CD₃; and R² is CH₂D.

In some embodiments, Y¹ is deuterium.

In some embodiments, Y^{2a}, Y^{2b}, Y^{3a} and Y^{3b} are the same. In particular embodiments, Y^{2a}, Y^{2b}, Y^{3a} and Y^{3b} are each deuterium.

In some embodiments, Y^{2a} and Y^{2b} are the same or different. In some aspects of these embodiments, Y^{2a} and Y^{2b} are each deuterium. In other aspects of these embodiments, Y^{2a} is hydrogen and Y^{2b} is deuterium.

In some embodiments, Y^{3a} and Y^{3b} are the same or different. In some aspects of these embodiments, Y^{3a} and Y^{3b} are each deuterium. In other aspects of these embodiments, Y^{3a} is hydrogen and Y^{3b} is deuterium.

In some embodiments, Y¹, Y^{2a} and Y^{2b} are all deuterium; and Y^{3a} and Y^{3b} are each independently hydrogen or deuterium. In particular aspects of these embodiments, Y¹, Y^{2a}, Y^{2b} and Y^{3a} are all deuterium; and Y^{3b} is hydrogen.

In some embodiments, Y¹, Y^{2a} and Y^{2b} are all hydrogen; and Y^{3a} and Y^{3b} are each independently hydrogen or deuterium. In particular aspects of these embodiments, Y¹, Y^{2a}, Y^{2b} and Y^{3a} are all hydrogen and Y^{3b} is deuterium.

In some embodiments, Y⁴ is deuterium.

In some embodiments, Y⁵ is deuterium.

In some embodiments, Y⁶, Y⁷, Y⁸ and Y⁹ are the same. In some aspects of these embodiments, Y⁶, Y⁷, Y⁸ and Y⁹ are all deuterium.

In one embodiment, the compound of Formula A is not a compound wherein each of Y¹, Y^{2a}, Y^{2b}, Y^{3a}, Y^{3b}, Y⁴, Y⁵, Y⁶, Y⁷, Y⁸, and Y⁹ is deuterium; each of R^{1a}, R^{1b} and R^{1c} is —CD₃; and R² is —CD₃.

In certain of the embodiments of Formula A set forth above, any atom not designated as deuterium is present at its natural isotopic abundance.

In one embodiment R^{1a}, R^{1b} and R^{1c} are the same; Y¹, Y^{2a} and Y^{2b} are the same; Y^{3a} and Y^{3b} are the same; Y⁴ and Y⁵ are same or different; Y⁶, Y⁷, Y⁸ and Y⁹ are the same; R² is CD₃; and the compound is selected from any one of the compounds set forth in Table A below:

TABLE A
Exemplary embodiments of Formula A:

| | R^{1a}/ | | | | | |
|---|---|---|---|---|---|---|
| Compound | R^{1b}/R^{1c} | Y¹/Y^{2a}/Y^{2b} | Y^{3a}/Y^{3b} | Y⁴ | Y⁵ | Y⁶/Y⁷/Y⁸/Y⁹ |
| 101 | CH₃ | D | D | D | D | D |
| 102 | CD₃ | D | D | D | D | D |
| 103 | CHD₂ | D | D | D | D | D |
| 105 | CH₂D | D | D | D | D | D |
| 106 | CH₃ | H | D | D | D | D |
| 107 | CD₃ | H | D | D | D | D |
| 108 | CHD₂ | H | D | D | D | D |
| 109 | CH₂D | H | D | D | D | D |
| 110 | CH₃ | D | H | D | D | D |
| 111 | CD₃ | D | H | D | D | D |
| 112 | CHD₂ | D | H | D | D | D |
| 113 | CH₂D | D | H | D | D | D |
| 114 | CH₃ | D | D | H | D | D |
| 115 | CD₃ | D | D | H | D | D |
| 116 | CHD₂ | D | D | H | D | D |
| 117 | CH₂D | D | D | H | D | D |
| 118 | CH₃ | D | D | D | H | D |
| 119 | CD₃ | D | D | D | H | D |
| 120 | CHD₂ | D | D | D | H | D |
| 121 | CH₂D | D | D | D | H | D |
| 122 | CH₃ | D | D | D | D | H |
| 123 | CD₃ | D | D | D | D | H |
| 124 | CHD₂ | D | D | D | D | H |
| 125 | CH₂D | D | D | D | D | H |

or a pharmaceutically acceptable salt thereof, wherein any atom not designated as deuterium is present at its natural isotopic abundance.

In some embodiments, the compound of Formula A has the structure of Formula I.

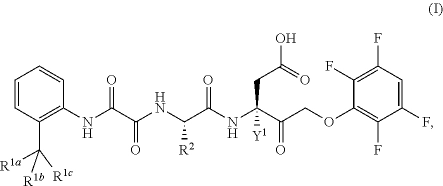

wherein

R^{1a}, R^{1b}, and R^{1c} are independently CH₃ or CD₃;

R² is CH₃ or CD₃;

Y¹ is hydrogen or deuterium; provided at least one of R^{1a}, R^{1b}, R^{1c}, R², and Y¹ comprises deuterium.

In some embodiments, at least one of R^{1a}, R^{1b} and R^{1c} is CD₃. In other embodiments, at least one R^{1a}, R^{1b} and R^{1c} is CH₃.

In some embodiments, R^{1a}, R^{1b}, and R^{1c} are all the same. In certain aspects of these embodiments, R^{1a}, R^{1b}, and R^{1c} are each CD₃. In certain aspects of these embodiments, R^{1a}, R^{1b}, and R^{1c} are each CH₃.

In some embodiments, R² is CD₃. In some embodiments, R² is CH₃.

In some embodiments, Y¹ is deuterium. In some embodiments, Y¹ is H.

In one embodiment, R^{1a}, R^{1b}, and R^{1c} are each CD₃; R² is CH3 or CD₃; and Y¹ is deuterium.

In certain of the embodiments of Formula I set forth above, any atom not designated as deuterium is present at its natural isotopic abundance.

In one embodiment, R^{1a}, R^{1b}, and R^{1c} are all the same, and the compound is selected from any one of the compounds (Cmpd) set forth in Table 1 (below):

TABLE 1
Exemplary Embodiments of Formula I

| Compound | R^{1a}/R^{1b}/R^{1c} | R² | Y¹ |
|---|---|---|---|
| 201 | CD₃ | CH₃ | H |
| 202 | CH₃ | CD₃ | H |
| 203 | CD₃ | CD₃ | H |
| 204 | CH₃ | CH₃ | D |
| 205 | CH₃ | CD₃ | D |
| 206 | CD₃ | CH₃ | D |
| 207 | CD₃ | CD₃ | D |

or a pharmaceutically acceptable salt thereof, wherein any atom not designated as deuterium is present at its natural isotopic abundance.

The synthesis of compounds of Formula A and/or Formula I may be readily achieved by synthetic chemists of ordinary skill by reference to the Exemplary Synthesis and Examples disclosed herein. Relevant procedures analogous to those of use for the preparation of compounds of Formula A and/or Formula I and intermediates thereof are disclosed, for instance in PCT Patent Application No. PCT/US99/15074 (published as WO200001666A1), or PCT Patent Application No. PCT/US02/01538 (published as WO2002057298A2), or PCT Patent Application No. PCT/IB07/03900 (published as WO2008068615A1).

Such methods can be carried out utilizing corresponding deuterated and optionally, other isotope-containing reagents and/or intermediates to synthesize the compounds delineated herein, or invoking standard synthetic protocols known in the art for introducing isotopic atoms to a chemical structure.

### Exemplary Synthesis

The compounds of Formula A and/or Formula I may be prepared in a manner analogous to that described in PCT Patent Application No. PCT/US99/15074 (published as WO200001666A1), or PCT Patent Application No. PCT/US02/01538 (published as WO2002057298A2), or PCT Patent Application No. PCT/IB07/03900 (published as WO2008068615A1) using appropriately deuterated starting materials which are commercially available or which synthesis is readily apparent to one of skill in the art.

A convenient method for synthesizing compounds of Formula A is depicted in Scheme 1 below.

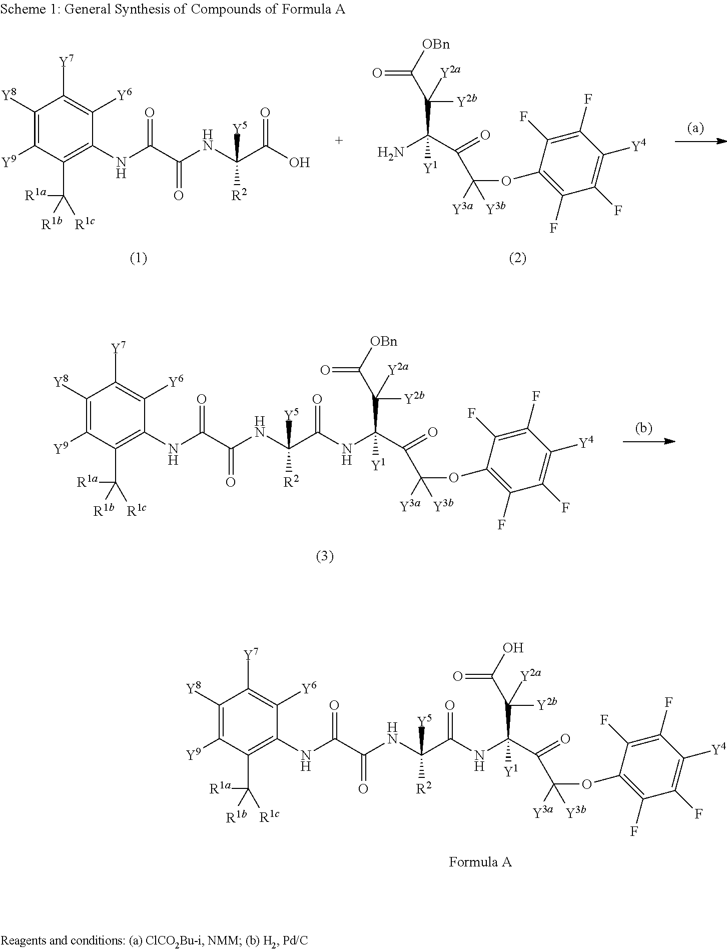

In a manner analogous to a procedure described in U.S. Pat. No. 7,692,038, mixed anhydride mediated coupling of appropriately deuterated acid intermediate (1) and benzyl protected amine intermediate (2) (optionally used as a salt), using a peptide coupling reagent such as isobutyl chloroformate in the presence of excess N-methylmorpholine (NMM) affords appropriately deuterated amide intermediate (3). Subsequent hydrogenolysis using palladium on activated charcoal in the presence of hydrogen gas produces appropriately deuterated compounds of Formula A.

Using commercially available reagents and deuterated reagents that can be readily prepared by known methods, compounds of Formula A can be prepared with greater than 90% or greater than 95% deuterium incorporation at each position designated as D (see below for details).

Appropriately deuterated intermediate (1), for use in the preparation of compounds of Formula A according to Scheme 1, may be prepared from corresponding deuterated reagents exemplified in Scheme 2 below.

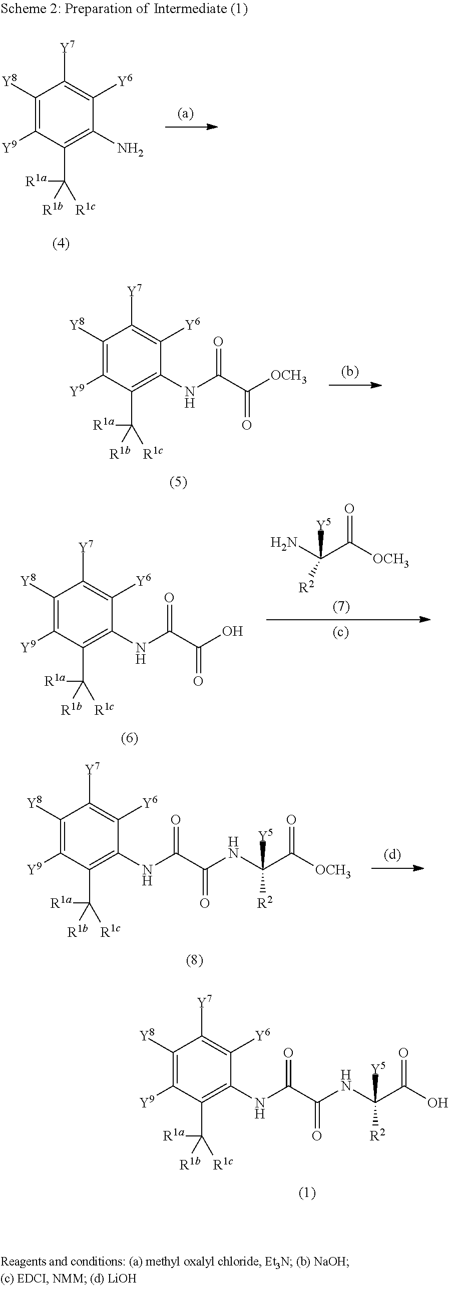

Analogous to a procedure described in U.S. Pat. No. 7,692,038, appropriately deuterated aniline intermediate (4) is treated with methyl oxalyl chloride in the presence of a base such as trimethylamine to furnish appropriately deuterated methyl ester intermediate (5), which is subsequently hydrolyzed using a base such as sodium hydroxide to afford appropriately deuterated acid intermediate (6). Coupling of (6) with appropriately deuterated amino acid ester intermediate (7) (optionally used as a salt) using a peptide coupling reagent such as 1-ethyl-3-(3-dimethylaminopropyl)carbodiimide (EDCI) in the presence of a base such as NMM furnishes appropriately deuterated methyl ester intermediate (8). Finally, hydrolysis with a base such as lithium hydroxide produces appropriately deuterated acid intermediate (1).

Intermediate (7) is commercially available and /or can be prepared by routine procedures within the common knowledge of a person of ordinary skills. For example, appropriately deuterated intermediates (7) can be prepared by such routine procedures from the following commercially available deuterated amino acids: L-alanine-2,3,3,3-d₄ (98 atom % D) (for intermediate (7a)), L-alanine-3,3,3-d3 (99 atom % D) (for intermediate (7b)), L-alanine-2-di(98 atom % D) (for intermediate (7c)).

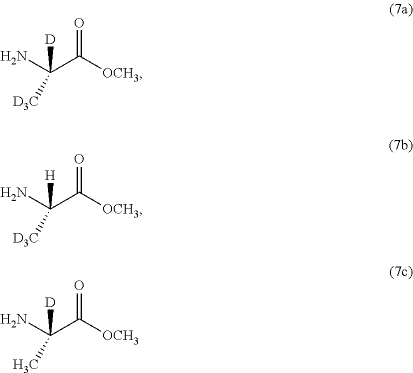

Use of appropriately deuterated reagents allows deuterium incorporation at any of the Y⁵, Y⁶, Y⁷, Y⁸, Y⁹, R^{1a}, R^{1b}, R^{1c}, and R² positions of a compound of Formula A or any appropriate intermediate herein, e.g., 90, 95, 97, or 99% deuterium incorporation at any Y⁵, Y⁶, Y⁷, Y⁸, Y⁹, R^{1a}, R^{1b}, R^{1c} and /or R².

Appropriately deuterated intermediate (2), for use in the preparation of compounds of Formula A according to Scheme 1, may be prepared from corresponding deuterated reagents exemplified in Scheme 3 below.

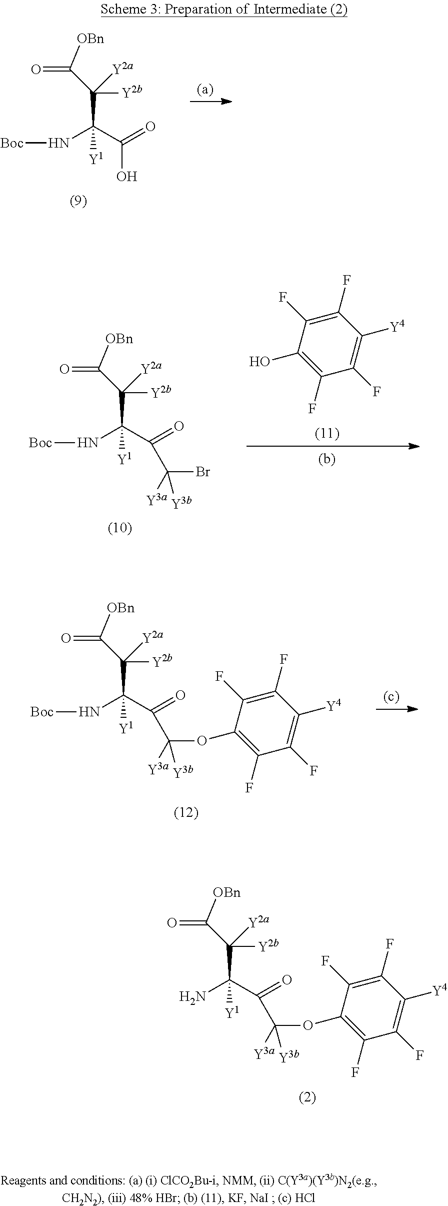

In a manner analogous to a procedure described in U.S. Pat. No. 7,692,038, or by Albeck, A. et al., Tetrahedron 1994, 50 (21), 6333-6346, and Albeck, A. et al., Tetrahedron 1997, 53 (14), 5325-5338, orthogonally protected and appropriately deuterated a-amino acid (9) is activated with an alkyl chloroformate such as isobutyl chloroformate to furnish a mixed anhydride, which is treated with diazomethane to provide a diazoketone. Subsequent treatment of the diazoketone with hydrobromic acid produces appropriately deuterated haloketone intermediate (10). Treatment of (10) with appropriately deuterated tetrafluorophenolate (11) by analogy to a procedure described by Ueno, H. et al., Bioorganic & Medicinal Chemistry Letters, 19(1), 199-202; 2009 or by Linton, S. et al., Journal of Medicinal Chemistry, 48(22), 6779-6782; 2005, affords Boc-protected and appropriately deuterated tetrafluoroaryloxy methylketone intermediate (12). Finally, treatment with acid such as HCl produces benzyl protected and appropriately deuterated amine intermediate (2).

Intermediate (9) is commercially available and /or can be prepared by routine procedures within the general knowledge of a person of ordinary skills. For example, appropriately deuterated intermediates (9) can be prepared by from the following commercially available deuterated amino acids: L-aspartic-2,3,3-d₃ acid (98 atom % D) (for intermediate (9a)); L-aspartic-3,3-d2 acid (for intermediate (9b)) for use may be prepared according to a procedure described by Zhang, S. et al., Journal of Peptide Research (2000), 55(1), 1-6; L-aspartic-2-d acid (for intermediate (9c)) for use may be prepared according to a procedure described by Taglang, C. et al., Angewandte Chemie, International Edition, 54(36), 10474-10477; 2015.

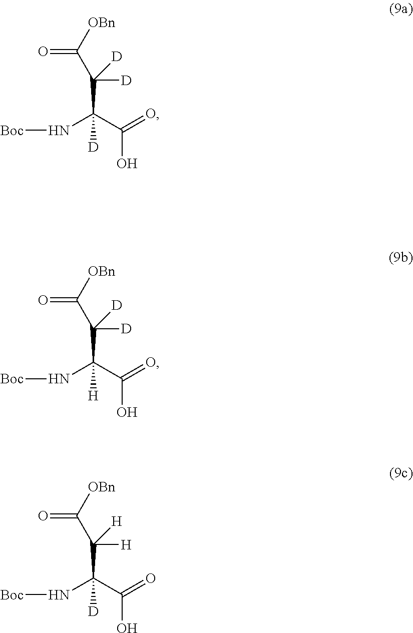

Intermediate (11a, Y⁴=D) is prepared by analogy to a procedure described by Colomban, C. et al., Journal of the American Chemical Society, 136(32), 11321-11330; 2014, from benzene-d, 2,3,4,5,6-pentafluoro-, which in turn is prepared according to a procedure described by Xiao, J. et al., Organic Letters, 17(4), 812-815; 2015. [96] Use of appropriately deuterated reagents allows deuterium incorporation at the Y¹, Y^{2a}, Y^{2b}, Y^{3a,3b}, and Y⁴ positions of a compound of Formula A or any appropriate intermediate herein, e.g., 90, 95, 97, or 99% deuterium incorporation at any Y¹, Y^{2a,2b}, Y^{3a,3b}, and /or Y⁴.

Appropriately deuterated intermediate (4), for use in the preparation of compounds of Formula A, according to Scheme 1 may be prepared from corresponding deuterated reagents exemplified in Scheme 4 below.

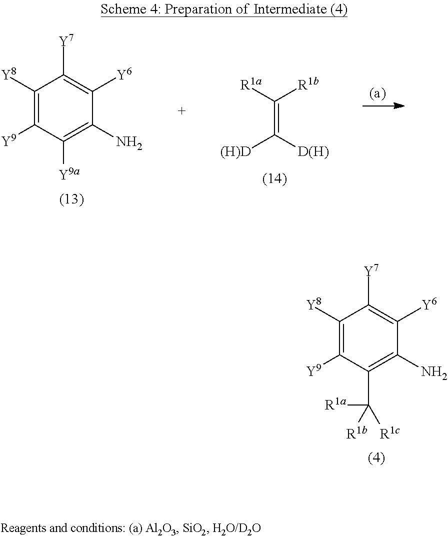

By analogy to a procedure described in EP0336134, appropriately deuterated mono-, and ortho-substituted aniline (4) is produced from appropriately deuterated aniline intermediate (13) and appropriately deuterated isobutylene intermediate (14) using silica-alumina catalysts.

Fully deuterated aniline intermediate (13a, wherein each of Y⁶, Y⁷, Y⁸ Y⁹ is deuterium) is commercially available and may be selected from: aniline-2,3,4,5,6-d5 (99 atom % D) or aniline-d7 (98 atom % D).

The following intermediates (14) are commercially available: 2-methylpropene-d₈ (99 atom % D) (14a); 1-propene-3,3,3-d3,2-(methyl-d3)-(98 atom % D) (14b); 1-propene-3,3,3-d3, 2-methyl-(14c) may be prepared according to a procedure described by Orfanopoulos, M. et al., Journal of the American Chemical Society, 112(9), 3607-14; 1990.

Use of appropriately deuterated reagents allows deuterium incorporation at the Y⁶, Y⁷, Y⁸, Y⁹, R^{1a}, R^{1b} and R^{1c} positions of a compound of Formula A or any appropriate intermediate herein, e.g., 90, 95, 97, or 99% deuterium incorporation at Y⁶, Y⁷, Y⁸, Y⁹, R^{1a}, R^{1b} and/or R^{1c}.

Additional methods of synthesizing compounds of Formula A and/or Formula I and their synthetic precursors, are within the means of chemists of ordinary skill in the art. Synthetic chemistry transformations and protecting group methodologies (protection and deprotection) useful in synthesizing the applicable compounds are known in the art and include, for example, those described in Larock R, Comprehensive Organic Transformations, VCH Publishers (1989); Greene, T W et al., Protective Groups in Organic Synthesis, 3^{rd} Ed., John Wiley and Sons (1999); Fieser, L et al., Fieser and Fieser's Reagents for Organic Synthesis, John Wiley and Sons (1994); and Paquette, L, ed., Encyclopedia of Reagents for Organic Synthesis, John Wiley and Sons (1995) and subsequent editions thereof.

Combinations of substituents and variables envisioned by this invention are only those that result in the formation of stable compounds.

### Compositions

The invention also provides pharmaceutical compositions comprising an effective amount of a compound of Formula A and/or Formula I (e.g., including any of the formulae herein), or a pharmaceutically acceptable salt of said compound; and a pharmaceutically acceptable carrier. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present invention in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound of this invention optionally formulated with a poloxamer, such as LUTROL™ and PLURONIC™ (BASF Corporation), or block copolymers of ethylene oxide and propylene oxide. See U.S. Pat. No. 7,014,866; and Untied States patent publications 20060094744 and 20060079502.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, Md. (20th ed. 2000).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In certain embodiments, the compound is administered orally. Compositions of the present invention suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, e.g.: Rabinowitz J D and Zaffaroni A C, U.S. Pat. No. 6,803,031, assigned to Alexza Molecular Delivery Corporation.

Topical administration of the pharmaceutical compositions of this invention is especially useful when the desired treatment involves areas or organs readily accessible by topical application. For topical application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax, and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol, and water. The pharmaceutical compositions of this invention may also be topically applied to the lower intestinal tract by rectal suppository formulation or in a suitable enema formulation. Topically-transdermal patches and iontophoretic administration are also included in this invention.

Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access.

Thus, according to yet another embodiment, the compounds of this invention may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the invention provides a method of coating an implantable medical device comprising the step of contacting said device with the coating composition described above. It will be obvious to those skilled in the art that the coating of the device will occur prior to implantation into a mammal.

According to another embodiment, the invention provides a method of impregnating an implantable drug release device comprising the step of contacting said drug release device with a compound or composition of this invention. Implantable drug release devices include, but are not limited to, biodegradable polymer capsules or bullets, non-degradable, diffusible polymer capsules and biodegradable polymer wafers.

According to another embodiment, the invention provides an implantable medical device coated with a compound or a composition comprising a compound of this invention, such that said compound is therapeutically active.

According to another embodiment, the invention provides an implantable drug release device impregnated with or containing a compound or a composition comprising a compound of this invention, such that said compound is released from said device and is therapeutically active.

Where an organ or tissue is accessible because of removal from the subject, such organ or tissue may be bathed in a medium containing a composition of this invention, a composition of this invention may be painted onto the organ, or a composition of this invention may be applied in any other convenient way.

In another embodiment, a composition of this invention further comprises a second therapeutic agent. The second therapeutic agent may be selected from any compound or therapeutic agent known to have or that demonstrates advantageous properties when administered with a compound having the same mechanism of action as emricasan. Such agents include those indicated as being useful in combination with emricasan, including but not limited to, those described in a PCT Patent Application No. PCT/US2008/002592 (published as WO2008106167A1), or a PCT Patent Application No. PCT/US2009/054292 (published as WO2010022140A1), or a PCT Patent Application No. PCT/US2012/061324 (published as WO2013059791A2).

Preferably, the second therapeutic agent is an agent useful in the treatment of a disease or condition selected from inflammatory conditions (e.g. arthritis, cholangitis, colitis, encephalitis, endocerolitis, pancreatitis, reperfusion injury, septic shock, septicemia, lens scarring and adult respiratory distress syndrome); immune-based diseases (e.g. hypersensitivity); autoimmune diseases (e.g., rheumatoid arthritis, systemic lupus erythematosus, scleroderma, chronic thyroiditis, Graves' disease, autoimmune gastritis, autoimmune hemolytic anemia, autoimmune neutropenia, thrombocytopenia, myasthenia gravis and multiple sclerosis); neurodegenerative diseases (e.g. primary lateral sclerosis, amyotrophic lateral sclerosis (ALS/Lou Gehrig's Disease), Alzheimer's disease, Parkinson's disease, diabetic neuropathy, Fahr's disease, Menke's disease, Wilson's disease, cerebral ischemia, a prion disorder (e.g., Creutzfeldt-Jakob disease), dementia (e.g., frontotemporal dementia, dementia with lewy bodies), corticobasal degeneration, progressive supranuclear palsy, multiple system atrophy, hereditary spastic paraparesis, and spinocerebellar atrophies); acute inflammation comprising the immediate and early response to an injurious agent (e.g. chemical agents, such as cosmetic agents and plant or insect derived agents, physical trauma, burns, or transient microbiologic infections); chronic inflammation, such as that resulting from the continued presence of injurious stimuli (e.g. persistent infections by intracellular micro-organisms, prolonged exposure to nondegradable inanimate material, and autoimmune diseases); inflammation or sepsis caused by a variety of infections (e.g. bacterial, fungal, and viral infections); infectious diseases (e.g. meningitis and salpingitis); bone diseases; polyglutamine (polyQ) diseases; respiratory diseases; wound healing; kidney diseases; digestive system diseases; low population of hematopoietic cells of a patient following radio- or chemotherapy, short term viability of organs to be used in transplantations (e.g. liver transplantation); diabetes (e.g. type II diabetes (through the reduction of insulin resistance), insulin-dependent diabetes mellitus); viral infections (e.g. hepatitis B and hepatitis C); hepatitis (e.g. alcoholic hepatitis, acute alcoholic hepatitis, lobular hepatitis, chronic Hepatitis C, chronic active hepatitis, autoimmune hepatitis); fibrosis (e.g. liver fibrosis, hepatic fibrosis); liver diseases (e.g. alcoholic liver disease, non-alcoholic fatty liver disease, drug-induced liver disease, acute liver disease; chronic liver disease); cirrhosis (e.g. liver cirrhosis, hepatic cirrhosis, primary biliary cirrhosis); liver impairment; periportal bridging necrosis; liver necrosis; hemochromatosis; sclerosing cholangitis; liver schistosomiasis; steatohepatitis; portal hypertension, non-alcoholic steatohepatitis, renal impairment, renal insufficiency; hepatic impairment; acute or chronic hepatic failure; acute liver failure; cholestasis; and hepatocellular carcinoma.

In a particular embodiment, the second therapeutic agent is a conventional anti-inflammatory agent, a matrix metalloprotease inhibitor, a lipoxygenase inhibitor, an antagonist of cytokines other than IL-1β, an immunomodulator (e.g., bropirimine, anti-human alpha interferon antibody, IL-2, GM-CSF, methionine enkephalin, interferon alpha, dietbyldithiocarbamate, tumor necrosis factor, naltrexons and rEPO), a prostaglandin, an anti-viral agent (e.g. ribavirin and interferon), a CCR-5 antagonist, an insulin sensitizer (e.g. metformin), hepatoprotectant (e.g. vitamin E, pentoxiphylline, betaine and ursodeoxycholic acid), a lipid-lowering agent (e.g. accomplia, orlistat, fibrate and cholestyramine), an HMG-CoA reductase inhibitor (e.g. atorvastatin), a glitastone, a biological agent (e.g. anti-TNFα antibody or an anti-MAdCAM antibody), an immunosuppressant (e.g. cyclosporine or tacrolimus), or a necrosis inhibitor (e.g. necrostatin, necrostatin-1, necrostatin-2, necrostatin-3, necrostatin-4, necrostatin-5, necrostatin-6 and necrostatin-7), a dopamine agonist of the non-ergoline class (e.g. pramipexole).

In another embodiment, the invention provides separate dosage forms of a compound of this invention and one or more of any of the above-described second therapeutic agents, wherein the compound and second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, the compound of the present invention is present in an effective amount. As used herein, the term “effective amount” refers to an amount which, when administered in a proper dosing regimen, is sufficient to treat the target disorder.

The interrelationship of dosages for animals and humans (based on milligrams per meter squared of body surface) is described in Freireich et al., Cancer Chemother. Rep, 1966, 50: 219. Body surface area may be approximately determined from height and weight of the subject. See, e.g., Scientific Tables, Geigy Pharmaceuticals, Ardsley, N.Y., 1970, 537.

In one embodiment, an effective amount of a compound of this invention can range from about 10 mg to about 100 mg per day. In some embodiments, an effective amount of a compound of this invention can range from about 2 mg per day to about 200 mg per day. In some embodiments, an effective amount of a compound of this invention can range from about 0.2 mg per day to about 1000 mg per day. In some embodiments, an effective amount of a compound of this invention can range from about 0.002 mg per day to about 10 grams per day. In certain embodiments, an effective amount of the compound is from about 10 mg to about 100 mg per day, administered in two equal doses per day (e.g., two doses of 5 mg each, two doses of 25 mg each, or two doses of 50 mg each).

Effective doses will also vary, as recognized by those skilled in the art, depending on the diseases treated, the severity of the disease, the route of administration, the sex, age and general health condition of the subject, excipient usage, and the possibility of co-usage with other therapeutic treatments such as use of other agents and the judgment of the treating physician. For example, guidance for selecting an effective dose can be determined by reference to the prescribing information for Emricasan.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

It is expected that some of the second therapeutic agents referenced above will act synergistically with the compounds of this invention. When this occurs, it will allow the effective dosage of the second therapeutic agent and/or the compound of this invention to be reduced from that required in a monotherapy. This has the advantage of minimizing toxic side effects of either the second therapeutic agent of a compound of this invention, synergistic improvements in efficacy, improved ease of administration or use and/or reduced overall expense of compound preparation or formulation.

### Methods of Treatment

In another embodiment, the invention provides a method of modulating the activity of interleukin-10 converting enzyme and related proteases (ICE/ced-3 family of cysteine proteases). In a particular embodiment, the invention provides a method of modulating the activity of one or more caspase proteases in a cell, comprising contacting a cell with one or more compounds of Formula A and/or Formula I herein, or a pharmaceutically acceptable salt thereof. In some embodiments, the one or more caspase proteases is selected from caspase-1, caspase-2, caspase-3, caspase-4, caspase-5, caspase-6, caspase-7, caspase-8, caspase-9, caspase-10, caspase-11 and caspase-12. In some aspects of these embodiments, a method of modulating the activity of one or more caspase proteases is a method of inhibiting the activity of caspase-3 or caspase-7.

According to another embodiment, the invention provides a method of treating a disease that is beneficially treated by emricasan in a subject in need thereof, comprising the step of administering to the subject an effective amount of a compound or a composition of this invention. In one embodiment the subject is a patient in need of such treatment. Such diseases are well known in the art and are disclosed in, but not limited to the following patents and published applications: PCT Patent Application No. PCT/US99/15074 (published as WO200001666A1); PCT Patent Application No. PCT/US02/01538 (published as WO2002057298A2); PCT Patent Application No. PCT/I1307/03900 (published as WO2008068615A1); PCT Patent Application No. PCT/US2008/002592 (published as WO2008106167A1); PCT Patent Application No. PCT/US2009/054292 (published as WO2010022140A1); PCT Patent Application No. PCT/US2012/061324 (published as WO2013059791A2); and PCT Patent Application No. PCT/US03/04457 (published as WO2003068242A1). Such diseases include, but are not limited to inflammatory conditions (e.g. arthritis, cholangitis, colitis, encephalitis, endocerolitis, pancreatitis, reperfusion injury, septic shock, septicemia, lens scarring and adult respiratory distress syndrome); immune-based diseases (e.g. hypersensitivity); autoimmune diseases (e.g., rheumatoid arthritis, systemic lupus erythematosus, scleroderma, chronic thyroiditis, Graves' disease, autoimmune gastritis, autoimmune hemolytic anemia, autoimmune neutropenia, thrombocytopenia, myasthenia gravis and multiple sclerosis); neurodegenerative diseases (e.g. primary lateral sclerosis, amyotropic lateral sclerosis (ALS/Lou Gehrig's Disease), Alzheimer's disease, Parkinson's disease, diabetic neuropathy, Fahr's disease, Menke's disease, Wilson's disease, cerebral ischemia, a prion disorder (e.g., Creutzfeldt-Jakob disease), dementia (e.g., frontotemporal dementia, dementia with lewy bodies), corticobasal degeneration, progressive supranuclear palsy, multiple system atrophy, hereditary spastic paraparesis, and spinocerebellar atrophies); acute inflammation comprising the immediate and early response to an injurious agent (e.g. chemical agents, such as cosmetic agents and plant or insect derived agents, physical trauma, burns, or transient microbiologic infections); chronic inflammation, such as that resulting from the continued presence of injurious stimuli (e.g. persistent infections by intracellular micro-organisms, prolonged exposure to nondegradable inanimate material, and autoimmune diseases); inflammation or sepsis caused by a variety of infections (e.g. bacterial, fungal, and viral infections); infectious diseases (e.g. meningitis and salpingitis); bone diseases; polyglutamine (polyQ) diseases; respiratory diseases; wound healing; kidney disease; kidney diseases; digestive system diseases; low population of hematopoietic cells of a patient following radio- or chemotherapy, short term viability of organs to be used in transplantations (e.g. liver transplantation); diabetes (e.g.type II diabetes (through the reduction of insulin resistance), insulin-dependent diabetes mellitus); viral infections (e.g. hepatitis B and hepatitis C); hepatitis (e.g. alcoholic hepatitis, acute alcoholic hepatitis, lobular hepatitis, chronic Hepatitis C, chronic active hepatitis, autoimmune hepatitis); fibrosis (e.g. liver fibrosis, hepatic fibrosis); liver diseases (e.g. alcoholic liver disease, non-alcoholic fatty liver disease, drug-induced liver disease, acute liver disease; chronic liver disease); cirrhosis (e.g. liver cirrhosis, hepatic cirrhosis, primary biliary cirrhosis); liver impairment; periportal bridging necrosis; liver necrosis; hemochromatosis; sclerosing cholangitis; liver schistosomiasis; steatohepatitis; portal hypertension, non-alcoholic steatohepatitis, renal impairment, renal insufficiency; hepatic impairment; acute or chronic hepatic failure; acute liver failure; cholestasis; and hepatocellular carcinoma.

In one particular embodiment, the method of this invention is used to treat a disease or condition selected from cirrhosis (e.g. liver cirrhosis, hepatic cirrhosis), portal hypertension, hepatitis (e.g. alcoholic hepatitis, acute alcoholic hepatitis), non-alcoholic steatohepatitis, liver diseases (e.g. non-alcoholic fatty liver disease), fibrosis (e.g. liver fibrosis, hepatic fibrosis), renal impairment, renal insufficiency, kidney diseases, hepatic impairment, digestive system diseases, diabetes, acute or chronic hepatic failure, acute liver failure, liver transplantation, cholestasis, hepatocellular carcinoma, chronic Hepatitis C in a subject in need thereof.

Identifying a subject in need of such treatment can be in the judgment of a subject or a health care professional and can be subjective (e.g. opinion) or objective (e.g. measurable by a test or diagnostic method).

In another embodiment, any of the above methods of treatment comprises the further step of co-administering to the subject in need thereof one or more second therapeutic agents. The choice of second therapeutic agent may be made from any second therapeutic agent known to be useful for co-administration with emricasan. The choice of second therapeutic agent is also dependent upon the particular disease or condition to be treated. Examples of second therapeutic agents that may be employed in the methods of this invention are those set forth above for use in combination compositions comprising a compound of this invention and a second therapeutic agent.

The term “co-administered” as used herein means that the second therapeutic agent may be administered together with a compound of this invention as part of a single dosage form (such as a composition of this invention comprising a compound of the invention and an second therapeutic agent as described above) or as separate, multiple dosage forms. Alternatively, the additional agent may be administered prior to, consecutively with, or following the administration of a compound of this invention. In such combination therapy treatment, both the compounds of this invention and the second therapeutic agent(s) are administered by conventional methods. The administration of a composition of this invention, comprising both a compound of the invention and a second therapeutic agent, to a subject does not preclude the separate administration of that same therapeutic agent, any other second therapeutic agent or any compound of this invention to said subject at another time during a course of treatment.

Effective amounts of these second therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment of the invention, where a second therapeutic agent is administered to a subject, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

In yet another aspect, the invention provides the use of a compound of Formula A and/or Formula I alone or together with one or more of the above-described second therapeutic agents in the manufacture of a medicament, either as a single composition or as separate dosage forms, for treatment in a subject of a disease, disorder or symptom set forth above. Another aspect of the invention is a compound of Formula A and/or Formula I for use in the treatment in a subject of a disease, disorder or symptom thereof delineated herein.

### EXAMPLE 1

### Evaluation of Metabolic Stability

Microsomal Assay: Human liver microsomes (20 mg/mL) are obtained from Xenotech, LLC (Lenexa, Kans.). β-nicotinamide adenine dinucleotide phosphate, reduced form (NADPH), magnesium chloride (MgCl₂), and dimethyl sulfoxide (DMSO) are purchased from Sigma-Aldrich.

Determination of Metabolic Stability: 7.5 mM stock solutions of test compounds are prepared in DMSO. The 7.5 mM stock solutions are diluted to 12.5-50 μM in acetonitrile (ACN). The 20 mg/mL human liver microsomes are diluted to 0.625 mg/mL in 0.1 M potassium phosphate buffer, pH 7.4, containing 3 mM MgCl₂. The diluted microsomes are added to wells of a 96-well deep-well polypropylene plate in triplicate. A 10 μL aliquot of the 12.5-50 μM test compound is added to the microsomes and the mixture is pre-warmed for 10 minutes. Reactions are initiated by addition of pre-warmed NADPH solution. The final reaction volume is 0.5 mL and contains 0.5 mg/mL human liver microsomes, 0.25-1.0 μM test compound, and 2 mM NADPH in 0.1 M potassium phosphate buffer, pH 7.4, and 3 mM MgCl₂. The reaction mixtures are incubated at 37° C., and 50 μL aliquots are removed at 0, 5, 10, 20, and 30 minutes and added to shallow-well 96-well plates which contain 50 μL of ice-cold ACN with internal standard to stop the reactions. The plates are stored at 4° C. for 20 minutes after which 100 μL of water is added to the wells of the plate before centrifugation to pellet precipitated proteins. Supernatants are transferred to another 96-well plate and analyzed for amounts of parent remaining by LC-MS/MS using an Applied Bio-systems API 4000 mass spectrometer. The same procedure is followed for the non-deuterated counterpart of the compound of Formula A and/or Formula I and the positive control, 7-ethoxycoumarin (1 μM) Testing is done in triplicate.

Data analysis: The in vitro tins for test compounds are calculated from the slopes of the linear regression of % parent remaining (ln) vs incubation time relationship.

in vitro t_{1/2}=0.693/k

k=−[slope of linear regression of % parent remaining (ln) vs incubation time]

Data Analysis is Performed Using Microsoft Excel Software.

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention.

1. A compound of Formula A: 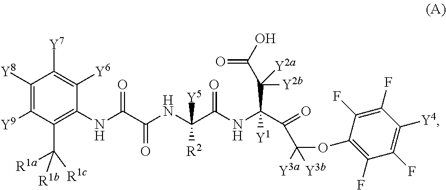
or a pharmaceutically acceptable salt thereof,
wherein
each of R^{1a}, R^{1b}, R^{1c} and R² is independently selected from CH₃ and CD₃;
each of Y¹, Y⁴ and Y⁵ is independently hydrogen or deuterium;
each of Y^{2a} and Y^{2b} is the same and is hydrogen or deuterium;
each of Y^{3a} and Y^{3b} is the same and is hydrogen or deuterium; and
each of Y⁶, Y⁷, Y⁸, and Y⁹ is the same and is hydrogen or deuterium;
provided that at least one of R^{1a}, R^{1b}, R^{1c}, R², Y¹, Y^{2a}, Y^{2b}, Y^{3a}, Y^{3b}, Y⁴, Y⁵, Y⁶, Y⁷, Y⁸ and Y⁹ comprises deuterium.

2. The compound of claim 1, wherein each of R^{1a}, R^{1b} and R^{1c} is CH₃.

3. The compound of claim 1, wherein each of R^{1a}, R^{1b} and R^{1c} is CD₃.

4. The compound of claim 1 wherein R² is CH₃.

5. The compound of claim 1, wherein R² is CD₃.

6. The compound of claim 1, wherein Y¹ is H.

7. The compound of claim 1, wherein Y¹ is D.

8. The compound of claim 1, wherein each of Y² a and Y^{2b} is H.

9. The compound of claim 1, wherein each of Y^{2a} and Y^{2b} is D.

10. The compound of claim 1, wherein each of Y^{3a} and Y^{3b} is H.

11. The compound of claim 1, wherein each of Y^{3a} and Y^{3b} is D.

12. The compound of claim 1, wherein Y⁴ is H.

13. (canceled)

14. The compound of claim 1, wherein Y⁵ is H.

15. The compound of claim 1, wherein Y⁵ is D.

16. The compound of claim 1, wherein each of Y⁶, Y⁷, Y⁸ and Y⁹ is H.

17. (canceled)

18. The compound of claim 1 wherein any atom not designated as deuterium is present at its natural isotopic abundance.

19. The compound of claim 1, wherein R^{1a}, R^{1b} and R^{1c} are the same; Y¹, Y^{2a} and Y^{2b} are the same; R² is CD₃; and the compound is selected from any one of the compounds set forth in the table below:

| | R^{1a}/ | | | | | |
|---|---|---|---|---|---|---|
| Compound | R^{1b}/R^{1c} | Y¹/Y^{2a}/Y^{2b} | Y^{3a}/Y^{3b} | Y⁴ | Y⁵ | Y⁶/Y⁷/Y⁸/Y⁹ |
| 101 | CH₃ | D | D | D | D | D |
| 102 | CD₃ | D | D | D | D | D |
| 106 | CH₃ | H | D | D | D | D |
| 107 | CD₃ | H | D | D | D | D |
| 110 | CH₃ | D | H | D | D | D |
| 111 | CD₃ | D | H | D | D | D |
| 114 | CH₃ | D | D | H | D | D |
| 115 | CD₃ | D | D | H | D | D |
| 118 | CH₃ | D | D | D | H | D |
| 119 | CD₃ | D | D | D | H | D |
| 122 | CH₃ | D | D | D | D | H |
| 123 | CD₃ | D | D | D | D | H |

or a pharmaceutically acceptable salt thereof, wherein any atom not designated as deuterium is present at its natural isotopic abundance.

20. A compound of Formula I: 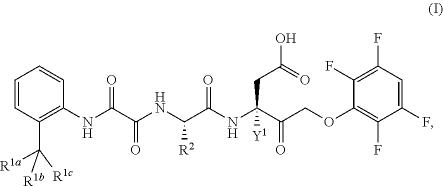
or a pharmaceutically acceptable salt thereof,
wherein
R^{1a}, R^{1b}, and R^{1c} are the same and are CH₃ or CD₃;
R² is CH₃ or CD₃;
Y¹ is hydrogen or deuterium;
provided at least one of R^{1a}, R^{1b}, R^{1c}, R^{2,} and Y¹ comprises deuterium.

21-24. (canceled)

25. The compound of claim 20, wherein any atom not designated as deuterium is present at its natural isotopic abundance.

26. The compound of claim 20, wherein the compound is selected from any one of the compounds set forth in the table below:

| Compound | R^{1a}/R^{1b}/R^{1c} | R² | Y¹ |
|---|---|---|---|
| 201 | CD₃ | CH₃ | H |
| 202 | CH₃ | CD₃ | H |
| 203 | CD₃ | CD₃ | H |
| 204 | CH₃ | CH₃ | D |
| 205 | CH₃ | CD₃ | D |
| 206 | CD₃ | CH₃ | D |
| 207 | CD₃ | CD₃ | D |

or a pharmaceutically acceptable salt thereof, wherein any atom not designated as deuterium is present at its natural isotopic abundance.

27. The compound of claim 1, wherein the deuterium incorporation at each designated deuterium atom is at least 90%.

28. A pharmaceutical composition comprising a compound of claim 1, or a pharmaceutically acceptable salt thereof, and a pharmaceutically acceptable carrier.

29. (canceled)

30. A method of treating a disease or condition selected from liver cirrhosis, hepatic cirrhosis, portal hypertension, hepatitis, alcoholic hepatitis, acute alcoholic hepatitis, non-alcoholic steatohepatitis, liver diseases, non-alcoholic fatty liver disease, liver fibrosis, hepatic fibrosis, renal impairment, renal insufficiency, kidney diseases, hepatic impairment, digestive system diseases, diabetes, acute or chronic hepatic failure, acute liver failure, liver transplantation, cholestasis, hepatocellular carcinoma and chronic Hepatitis C, comprising the step of administering to a subject in need thereof a compound of claim 1.

31. The compound of claim 27, wherein the deuterium incorporation at each designated deuterium atom is at least 95%.
